(19) United States
(12) Patent Application Publication
(10) Pub. No.: US 2013/0168507 A1
(43) Pub. Date: Jul. 4, 2013

(54) Endotracheal tube securing device and system

(71) Applicant: Shane E. Eastman, San Antonio, TX (US)

(72) Inventor: Shane E. Eastman, San Antonio, TX (US)

(73) Assignee: Shane E. Eastman, San Antonio, TX (US)

(21) Appl. No.: 13/374,562

(22) Filed: Jan. 3, 2012

(51) Int. Cl. A61B 19/00; A61M 16/04

(52) U.S. Cl. 248/65; 128/207.17

(57) ABSTRACT

A tube-securing device includes a flexible plate having multiple attachment points including multiple harness attachment points and multiple tube attachment points. A harness is attached to the flexible plate positioned to expose a patient's mouth and nose clear of the plate in response to adjusting the harness.

### BACKGROUND

The present disclosure relates generally to a medical device and system for securing a tube to a patient and more particularly to securing an endotracheal tube to a patient.

Often, during medical treatment, whether general or intensive, some endotracheal tube securing devices do not allow for alternating the position of the tube in the patient's mouth. This is a disadvantage because the likelihood of tissue damage and the resultant complications. Also, some devices block the airway or oral cavity which limits the ability to clean and suction unless the tube holding device is removed. Other devices may require the use of adhesive materials on the patient's skin, which can result in skin breakdown or irritation. Such devices cause severe problems, or may not be used, with burn victims. Certain devices which do include a position adjustable tube holder, are known to pinch the patient's mouth when an adjustment is being made. Accordingly, it would be beneficial to provide a tube securing device which avoids the above-mentioned disadvantages.

### SUMMARY

One embodiment of the present disclosure provides a tube securing device comprising a flexible plate having a plurality of attachment points including a plurality of harness attachment points and a plurality of tube attachment points. In addition, means are provided for maintaining the flexible plate positioned to expose a patient's mouth and nose clear of the plate in response to a harness being attached to the plate.

### BRIEF DESCRIPTION OF THE DRAWINGS

FIG. 1 is a frontal view illustrating an embodiment of a flexible plate.

FIG. 2 is a top view illustrating an embodiment of the flexible plate taken along the line 2-2 of FIG. 1.

FIG. 3 is a perspective view illustrating an embodiment of the flexible plate of FIG. 1.

FIG. 4 is a side view illustrating an embodiment of the flexible plate taken along the lines 4-4 of FIG. 1.

FIG. 5 is an enlarged perspective view illustrating an embodiment of a tube attachment point of the flexible plate.

FIG. 6 is an enlarged side view illustrating an embodiment of the tube attachment point having a tube secured to the tube attachment point by a piece of tape.

FIG. 7 is a plan view illustrating an embodiment of a portion of a harness.

FIG. 8 is a plan view illustrating an embodiment of another portion of the harness.

FIG. 9 is a frontal view illustrating an embodiment of a harness attached to a plate and adjusted to maintain the plate positioned adjacent a patient's nose and upper lip.

FIG. 10 is a rear view illustrating an embodiment of the harness positioned on the patient's head.

### DETAILED DESCRIPTION

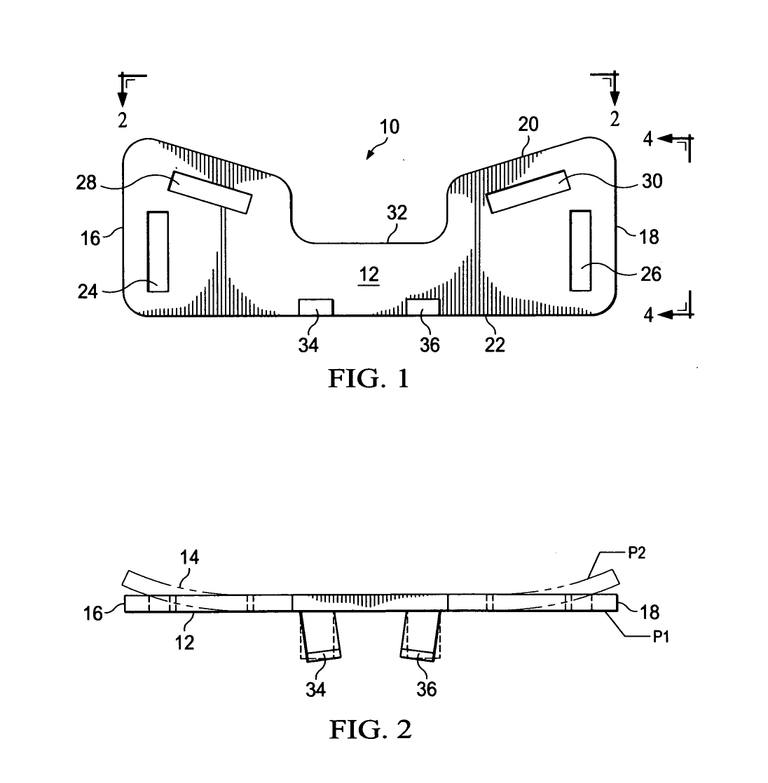

A flexible plate 10, FIGS. 1-6 is illustrated. Plate 10 is preferably formed of a suitable synthetic material, which can be formed into a flexible planar member. Plate 10 is substantially planar, position P1 in FIG. 2, but may be flexed to conform to a curvature of a patient's face in the area of the patient's nose and mouth, position P2 in FIG. 2.

The plate 10 includes a front surface 12 and a rear 14 surface, a pair of opposite edges 16, 18, an upper edge 20 and a lower edge 22.

A plurality of harness attachment points includes spaced apart apertures 24, 26, adjacent opposite edges 16, 18, respectively, and spaced apart apertures 28, 30 adjacent edge 20. A notch 32 is formed in edge 20 and is positioned between apertures 28, 30.

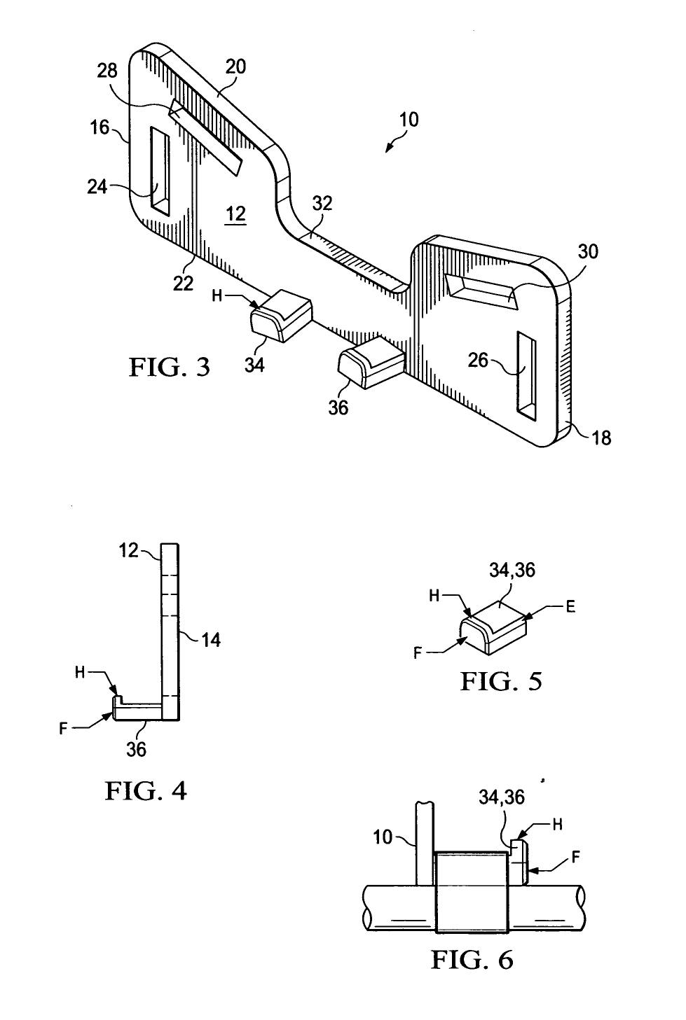

A plurality of fixed tube attachment points 34, 36 are formed as posts spaced apart along edge 22. The tube attachment points 34, 36 extend from a first end substantially perpendicularly disposed relative to surface 12 of plate 10. Points 34, 36 may be angled slightly toward each other, FIG. 2, to be discussed below. Edges E of the tube attachment points 34, 36 may be chamfered and a free end F, distal from the first end includes a raised hook H, to be discussed below, FIGS. 3-6.

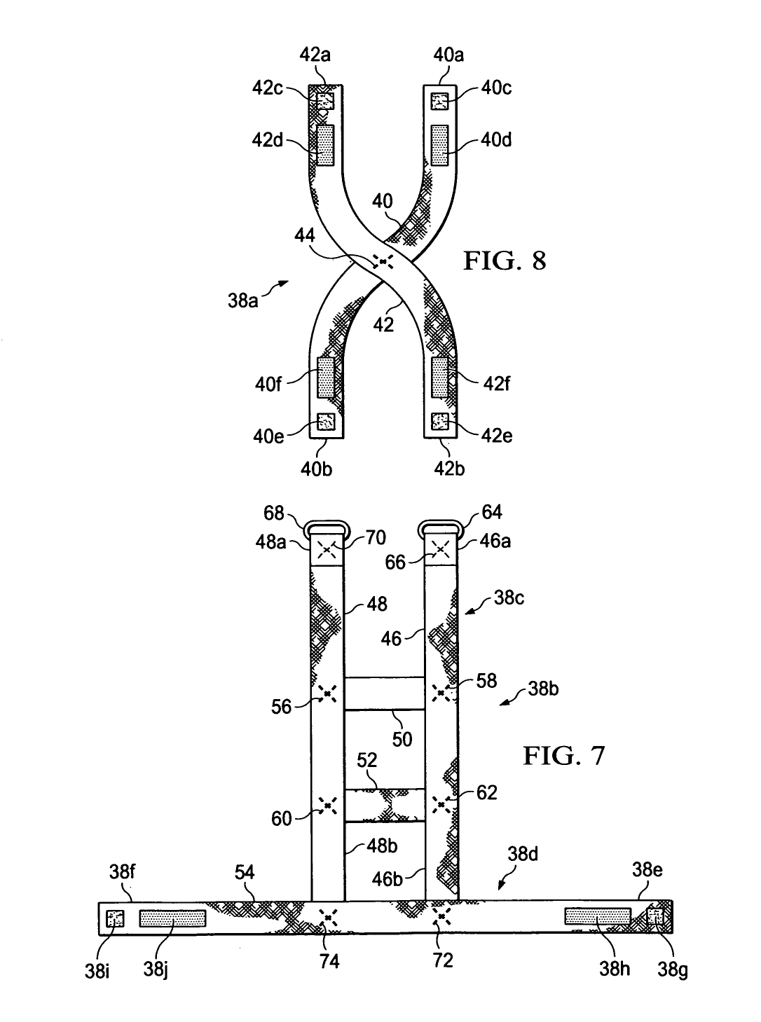

An adjustable harness, FIGS. 7-10, includes a first harness portion 38a and a second harness portion 38b. The first harness portion 38a is a generally X-shaped member formed of a suitable fabric for use on a patient's head and face. Portion 38a includes a first member 40 and a second member 42, which are attached together at stitches 44. First ends 40a, 42a of members 40, 42 respectively, are provided to attach to plate 10 by insertion through apertures 28, 30 respectively. Second ends 40b, 42b are also provided for an adjustable attachment to be discussed below. Attachment of first ends 40a, 42a is accomplished by inserting the ends 40a, 42a through apertures 28, 30 respectively, and folded so that, for example, a first connection portion 40c of member 40 is engaged with either of a second connector portion 40d, or engaged with the fabric of member 40, so that the second connector portion 40d may not be required. Such connector portions 40c, 40d may comprise a Velcro® material, for example. Similarly, a first connector portion 42c of member 42 is engaged with either of a second connector portion 42d, or engaged with the fabric of member 42 as set forth above.

The adjustable harness also includes the second harness portion 38b also formed of the same fabric as portion 38a. The second harness portion 38b is generally a ladder-type structure 38c attached to a base member 38d. The ladder-type structure 38c includes member 46, 48, 50 and 52. The base member 38d includes member 54. Members 46 and 48 are interconnected by means of member 50, 52 which attach by stitches 56, 58, 60, 62.

A loop member 64 is attached at an end 46a of member 46 by stitches 66. Another loop member 68 is attached at an end 48a of member 48 by stitches 70. Another end 46b of member 46 is attached to base member 38d by stitches 72 and an end 48b of member 48 is attached to base member 38d by stitches 74.

The adjustable harness portions 38a and 38b are joined by attaching end 42b of member 42 through loop 64 and attaching end 40b through loop 68. These attachments are accomplished by passing end 42b through loop 64 and folding end 42b so that a first connector portion 42e is engaged with of a second connector portion 42f or engaged with the fabric of member 42, so that the second connector portion of 42f may not be required. End 40b is passed through loop 68 so that first connector portion 40e is engaged with either of a second connector portion 40f or engaged with the fabric of member 40, so that the second connector portion 40f may not be required.

Base member 38d includes opposite ends 38e and 38f. End 38e includes connector member 38g and 38h, whereas end 38f includes connector members 38i and 38j. Base member 38b is adjustably attached to plate 10 by means of inserting end 38e through aperture 24 and inserting end 38f through aperture 26. The end 38e is folded so that connector portions 38g and 38h are engaged. The end 38f is similarly folded so that connector portions 38i and 38j are engaged. Alternatively, portions 38g and 38i may be secured by engagement to the fabric of member 38d.

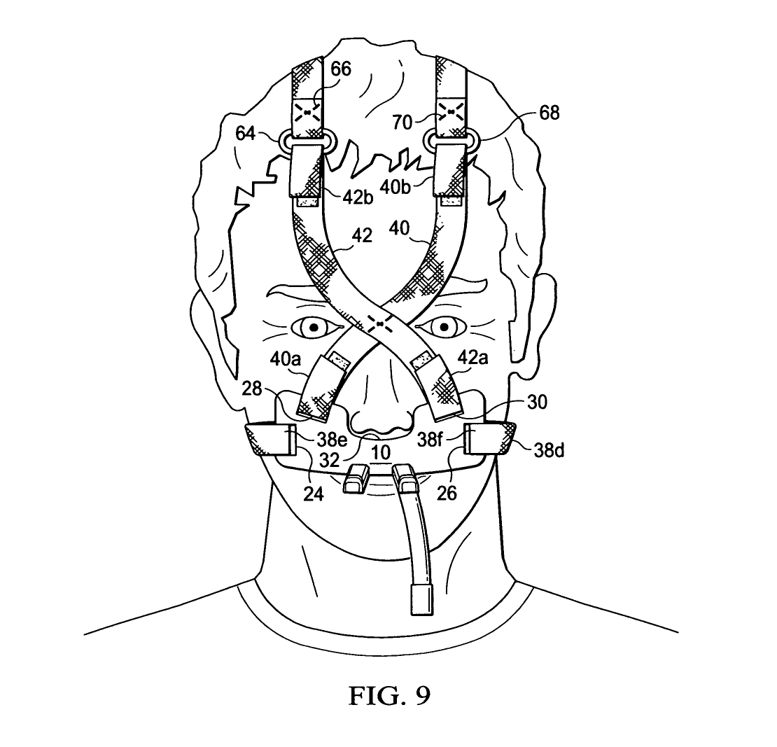

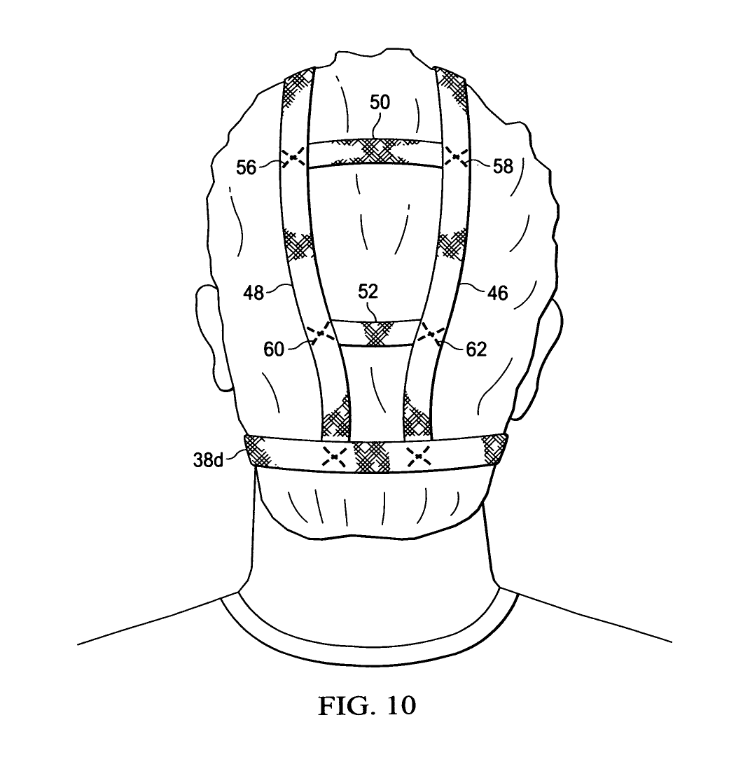

As can be seen, engagement of all of the connector portions described above are adjustably engaged with their respective mating connector portions, thus providing a wide rage of adjustment for positioning and maintaining plate 10 between a patient's nose and mouth so that airways are open and accessible as illustrated in FIGS. 9 and 10. Also, the plate 10 and harness portions 38a and 38b, although discussed independently above, may be pre-assembled so that a single attachment is required to secure the device to the patient's head, whereafter, adjustments at multiple connector portions, may be made for proper and comfortable fitting.

Advantages of this disclosed device include the provision of an easy-to-use device which can be quickly applied, adjusted and removed. The device, when properly adjusted, does not block any air passage, i.e. nose or mouth. The device is inexpensive to manufacture and provides improved comfort, stability and flexibility over currently available endotracheal tube securing devices. Use of this device enables a caregiver to adjust the device to meet the specific size requirement of an individual patient. Additionally, the device permits the alternating placement of the tube toward either side of the patient's mouth, so that prolonged placement of the tube can be varied to reduce the likelihood of tissue irritation and any resulting complications. The device does not have adhesive surfaces which can cause skin irritation or discomfort, nor is there any need to shave the patient prior to use. In the case of burn damage to a patient, skin or bandage contact is minimal. Also, a patient having no teeth, or requiring false teeth removal, presents no issues. Clearance of a patient's airway is possible even with the device in place.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A tube securing apparatus comprising:
a flexible plate;
a plurality of attachment points on the plate including:
a plurality of harness attachment points: and
a plurality of tube attachment points; and
means for maintaining the flexible plate positioned to expose a patient's nose and mouth clear of the plate in response to a harness being attached to the plate.

2. The apparatus as defined in claim 1 further comprising:
a notch formed in one edge of the flexible plate.

3. The apparatus as defined in claim 2 wherein the harness attachment points are in fixed, spaced apart positions on the flexible plate.

4. The apparatus as defined in claim 3 wherein the tube attachment points are in
fixed spaced apart positions on the flexible plate.

5. The apparatus as defined in claim 1 wherein the means for maintaining the position of the flexible plate includes:
a notch formed in one edge of the flexible plate;
the tube attachments points being fixed in position on the flexible plate;
the tube attachment plates being spaced apart and extending substantially perpendicularly from a second edge of the flexible plate opposite the one edge; and
a harness attached to the harness attachment points.

6. The apparatus as defined in claim 5 wherein the harness is adjustable to fit the patient's head size.

7. The apparatus as defined in claim 6 wherein adjustment of the harness maintains the notch adjacent the patient's nose and maintains the tube attachment points adjacent the patient's upper lip.

8. The apparatus as defined in claim 7 further comprising:
a tube movable for selective attachment to any one of the tube attachment points.

9. The apparatus as defined in claim 8 further comprising:
each tube attachment point having a first end attached to the flexible plate and a second free end distal from the first end; and
a raised lip formed on the second free end.

10. A tube securing system comprising:
a harness;
a flexible plate secured to the harness;
the flexible plate including a plurality of fixed harness attachment points and a plurality of fixed tube attachment points; and
means for maintaining the flexible plate positioned to expose a patient's mouth and nose clear of the plate in the response to the harness being attached to the plate.

11. The system as defined in claim 10 wherein the means for maintaining the position of the flexible plate includes:
a notch formed in one edge of the flexible plate;
the tube attachment points being spaced apart and extending substantially perpendicularly from a second edge of the flexible plate opposite the one edge;
the harness attachment points being in spaced apart positions on the flexible plate; and
the harness being attached to the harness attachment points.

12. The system as defined in claim 11 wherein the harness is adjustable to fit the patient's head size.

13. The system as defined in claim 12 wherein adjustment of the harness maintains the notch adjacent the patient's nose and maintains the tube attachment points adjacent the patient's upper lip.

14. The system defined as in claim 13 further comprising:
the tube being moveable for selective attachment to any one of the tube attachment points.

15. The system as defined in claim 14 further comprising:
each tube attachment point having a first end attached to the flexible plate and a second free end distal from the first end; and
a raised lip formed on the second end.

16. A tube securing apparatus comprising:
a flexible plate including a notch formed in one edge of the plate;
a plurality of fixed harness attachment points spaced apart on the flexible plate; and
a plurality of stationary tube attachment points spaced apart and extending substantially perpendicularly from a second edge of the flexible plate opposite the one edge.

17. The tube securing apparatus as defined in claim 16 further comprising:
a harness attached to the harness attachment points and a tube movable for selective attachment to any one of the tube attachment points.

18. The tube securing apparatus as defined in claim 17 wherein the harness is adjustable to fit a patient's head size.

19. The tube securing apparatus as defined in claim 18 wherein the harness is adjustable to maintain the notch adjacent the patient's nose and to maintain the tube attachment points adjacent the patient's upper lip.

20. The tube securing apparatus as defined in claim 19 comprising:
each tube attachment point having a first end attached to the flexible plate and a second free and distal from the first end; and
a raised lip formed on the second free end.
